(19) United States
(12) Patent Application Publication
(10) Pub. No.: US 2018/0045663 A1
(43) Pub. Date: Feb. 15, 2018

(54) MICRO SENSOR

(71) Applicant: POINT ENGINEERING CO., LTD., Asan (KR)

(72) Inventors: Bum Mo AHN, Suwon (KR); Seung Ho PARK, Hwaseong (KR); Sung Hyun BYUN, Asan (KR)

(21) Appl. No.: 15/674,127

(22) Filed: Aug. 10, 2017

(30) Foreign Application Priority Data
Aug. 11, 2016 (KR) 10-2016-0102340

(51) Int. Cl. G01N 27/12; G01N 33/00

(52) U.S. Cl. CPC G01N 27/128; G01N 33/0031; G01N 33/0016

(57) ABSTRACT

Disclosed is a micro sensor. Particularly, a first sensor electrode is provided on a first side of a substrate, a second sensor electrode is provided on a second side of the substrate, and the substrate is provided with an etching hole penetrating from the first side to the second side of the substrate. A sensing material provided between the first sensor electrode and the second sensor electrode is inserted in the etching hole. When detecting gas, the micro sensor can minimize influence of moisture in air.

### CROSS REFERENCE TO RELATED APPLICATION

The present application claims priority to Korean Patent Application No. 10-2016-0102340, filed Aug. 11, 2016, the entire contents of which is incorporated herein for all purposes by this reference.

### BACKGROUND OF THE INVENTION

### Field of the Invention

The present invention generally relates to a micro sensor. More particularly, the present invention relates to a micro sensor including a first sensor electrode formed on a first side of a substrate and a second sensor electrode formed on a second side of the substrate provided with an etching hole penetrating from the first side of the substrate to the second side of the substrate.

### Description of the Related Art

Recently, as concern about the environment is gradually increasing, there is a need for development of a micro sensor being capable of acquiring accurate and various pieces of information in a short period of time. Especially, for comfort of residential space, management of harmful industrial environment, management of foods and food production processes, etc., efforts have progressed in terms of realizing miniaturization, high-precision, and cost reduction of a micro multi-array sensor, such as a gas sensor, for easily measuring the relevant gas concentration.

Gas sensors are gradually evolving from conventional sintered ceramic or thick film structures into micro-electro-mechanical system (MEMS) gas sensors by applying semiconductor process technologies.

In terms of a measurement method, the most widely used method of a gas sensor today is measuring changes in electrical characteristics when the gas is adsorbed onto a sensing material of the sensor. Generally, metal oxides such as SnO₂ are used as a sensing material, and the change in the electrical conductivity thereof depending on the concentration of the target gas is measured. This measurement method is relatively simple. Here, the change in the measurement value is more significant when the metal oxide sensing material is operated at the high temperature. Therefore, precise temperature control is essential for fast and precise measurement of the gas concentration. Also, when measuring, the gas concentration is measured after resetting the sensing material to its initial state through high temperature heating, thereby forcibly removing gas species or moisture already adsorbed onto the sensing material.

However, a conventional sensor detects one kind of gas, and thus, several sensors are required to detect various kinds of gas, whereby the volume and power consumption increase.

Also, the sensing material is placed at the outside of the substrate and the measurement precision is low due to the moisture (humidity) in air.

The foregoing is intended merely to aid in the understanding of the background of the present invention, and is not intended to mean that the present invention falls within the purview of the related art that is already known to those skilled in the art.

### DOCUMENT OF RELATED ART

(Patent Document 1) Korean Patent Application Publication No. 10-2009-0064693.

### SUMMARY OF THE INVENTION

Accordingly, the present invention has been made keeping in mind the above problems occurring in the related art, and an object of the present invention is to provide a micro sensor being capable of minimizing the influence of moisture in air.

In order to accomplish the above object, the present invention provides a micro sensor including: a substrate; and a sensor electrode unit provided on the substrate, wherein the sensor electrode unit includes a first sensor electrode and a second sensor electrode spaced apart from the first sensor electrode, the first sensor electrode is provided on a first side of the substrate, the second sensor electrode is provided on a second side of the substrate, and the substrate is provided with an etching hole penetrating from the first side to the second side of the substrate.

The etching hole may be provided at a circumference of the first sensor electrode.

The second sensor electrode may block at least a part of the etching hole on the second side.

The first sensor electrode may include a first sensor wire, the second sensor electrode may include a second sensor wire, and an area of the second sensor wire may be larger than an area of the first sensor wire.

The first sensor electrode may include a first sensor wire and a first sensor electrode pad connected to the first sensor wire, the second sensor electrode may include a second sensor wire and a second sensor electrode pad connected to the second sensor wire, the first side of the substrate may be provided with an assistant pad, and the assistant pad may be connected to the second sensor electrode pad.

The substrate may be provided with a pad hole penetrating from the first side of the substrate to the second side of the substrate so as to connect the assistant pad and the second sensor electrode pad to each other.

The micro sensor may include a heater electrode unit provided on the first side of the substrate.

The substrate may be an anodized film obtained by anodizing a metallic base material and removing the base material.

The substrate may be provided with an air gap surrounding the heater electrode unit.

At least a part of a surface of the heater electrode unit may be provided with a passivation layer.

According to another aspect, there is provided a micro sensor including: a substrate; a sensor electrode unit provided on the substrate; and a heater electrode unit provided on the substrate, wherein the sensor electrode unit includes a first sensor electrode unit and a second sensor electrode unit, the heater electrode unit includes a first heater electrode unit having a first heating wire and a second heater electrode unit having a second heating wire, the first sensor electrode unit is closer to the first heating wire than to the second heating wire, the second sensor electrode unit is closer to the second heating wire than to the first heating wire, at least one of the first sensor electrode unit and the second sensor electrode unit includes a first sensor electrode and a second sensor electrode spaced apart from the first sensor electrode, the first sensor electrode is provided on a first side of the substrate, the second sensor electrode is provided on a second side of the substrate, and the substrate is provided with an etching hole penetrating from the first side of the substrate to the second side of the substrate.

The first heating wire and the second heating wire may have different heating values.

According to the micro sensor of the present invention as described above, effects as follows may be obtained.

The first sensor electrode is formed on the first side of the substrate and the second sensor electrode is formed on the second side of the substrate. The substrate is provided with the etching hole penetrating from the first side of the substrate to the second side of the substrate. A sensing material provided between the first sensor electrode and the second sensor electrode is inserted in the etching hole. When detecting gas, influence of moisture in air can be minimized, thereby enhancing measurement precision.

The first sensor electrode includes the first sensor wire and the first sensor electrode pad connected to the first sensor wire. The second sensor electrode includes the second sensor wire and the second sensor electrode pad connected to the second sensor wire. The first side of the substrate is provided with the assistant pad, and the assistant pad is connected to the second sensor electrode pad. Soldering of the first sensor electrode and the second sensor electrode can be performed on the same surface, and thus a soldering process is easier.

The substrate is provided with the pad hole penetrating from the first side of the substrate to the second side of the substrate so as to connect the assistant pad and the second sensor electrode pad, whereby the structure of the sensor can be simple.

The substrate is the anodized film obtained by anodizing the metallic base material and removing the base material, thereby enhancing insulation effect.

The substrate is provided with the air gap surrounding the heater electrode unit, whereby a heat capacity is small and high temperature can be maintained by low power.

At least a part of the surface of the heater electrode unit is provided with the passivation layer. Thus, the heater electrode unit can be electrically insulated from the sensing material, and the heater electrode unit can be protected from oxidation.

The first heating wire and the second heating wire of the sensor have different heating values. When applied to a gas sensor, various kinds of gas can be simultaneously detected with a simple structure.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a plan view showing a micro sensor according to an exemplary embodiment of the present invention (except for a sensing material and a passivation layer);

FIG. 2 is an enlarged view of A portion of FIG. 1;

FIG. 3 is an enlarged view of B portion of FIG. 1;

FIG. 4 is a plan view showing a micro sensor according to an exemplary embodiment of the present invention;

FIG. 5 is a bottom view showing a micro sensor according to an exemplary embodiment of the present invention; and

FIG. 6 is a cross-sectional view of C-C portion of FIG. 4.

### DETAILED DESCRIPTION OF THE INVENTION

Hereinbelow, exemplary embodiments of the present invention will be described in detail with reference to the accompanying drawings.

For reference, in the following descriptions, the same configurations of the present invention as those of the related art will not be described in detail. Reference is made to the foregoing descriptions of the related art.

When it is said that any part is positioned “on” another part, it means the part is directly on the other part or above the other part with at least one intermediate part. In contrast, if any part is said to be positioned “directly on” another part, it means that there is no intermediate part between the two parts.

Technical terms used here are to only describe a specific exemplary embodiment and are not intended to limit the present invention. Singular forms used here include a plurality of forms unless phrases explicitly represent an opposite meaning. A meaning of “comprising” used in a specification embodies a specific characteristic, area, integer, step, operation, element and/or component and does not exclude presence or addition of another specific characteristic, area, integer, step, operation, element, component and/or group.

Terms representing relative space of “low” and “upper” may be used for more easily describing a relationship to another portion of a portion shown in the drawings. Such terms are intended to include other meanings or operations of a using apparatus together with a meaning that is intended in the drawings. For example, when an apparatus is inverted in the drawings, any portion described as disposed at a “low” portion of other portions is described as being disposed at an “upper” portion of other portions. Therefore, an illustrative term of “low” includes entire upper and lower directions. An apparatus may rotate by 90° or another angle, and a term representing relative space is accordingly analyzed.

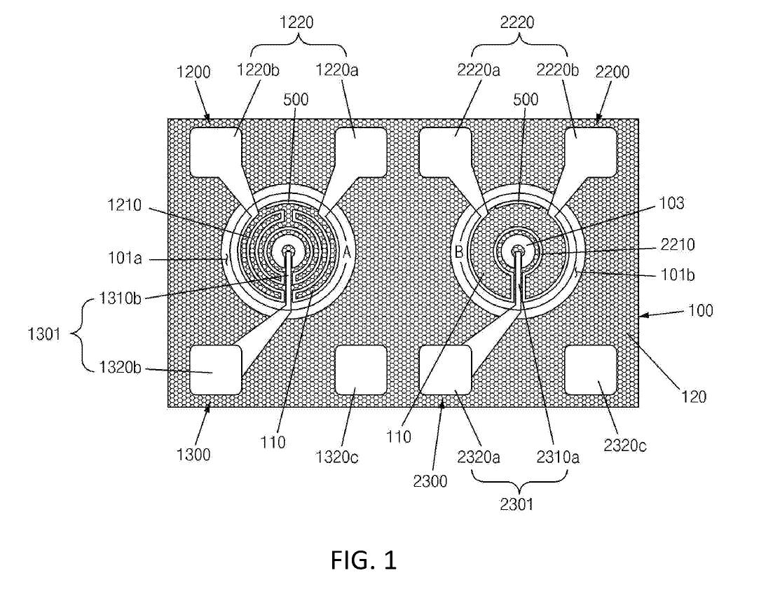

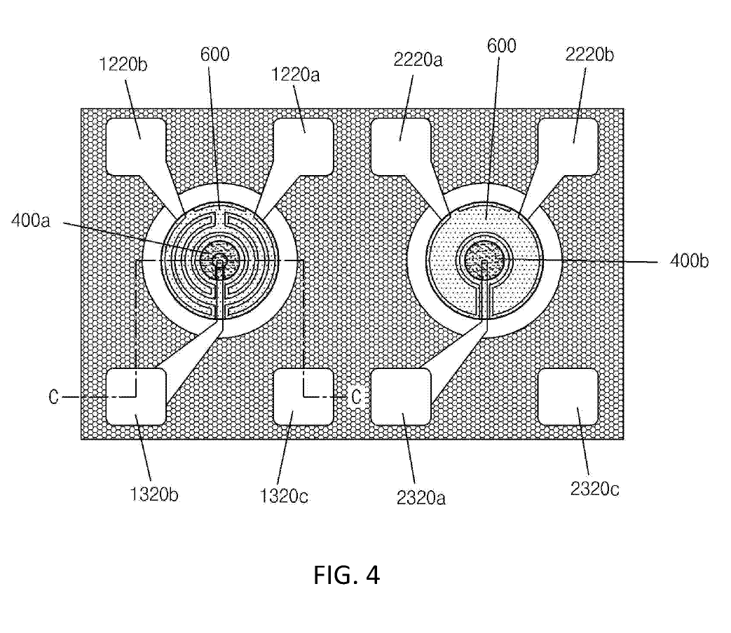

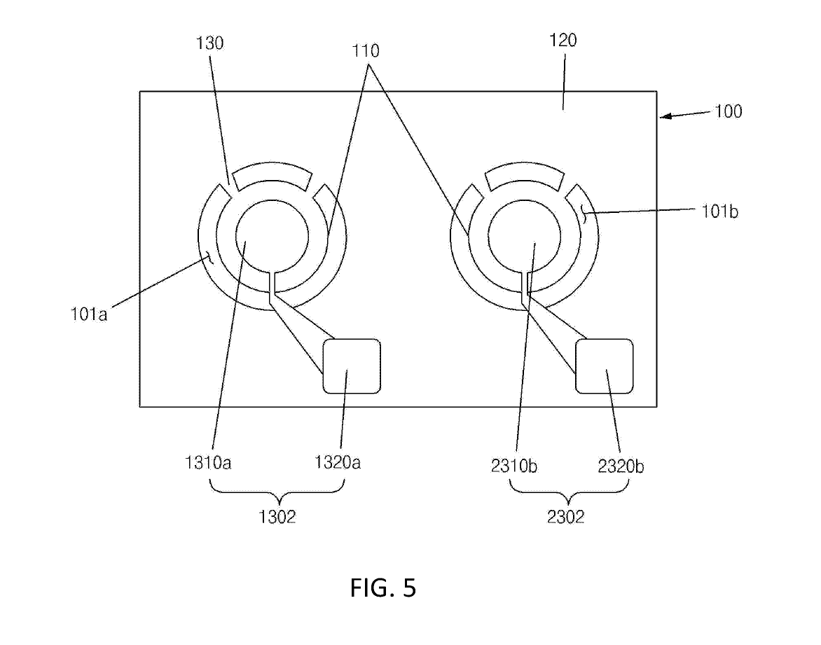

As shown in FIGS. 1 to 6, according to the embodiment, a micro sensor includes a substrate 100, a sensor electrode unit formed on the substrate 100, and a heater electrode unit formed on the substrate 100. The sensor electrode unit includes a first sensor electrode unit 1300 and a second sensor electrode unit 2300. The heater electrode unit includes a first heater electrode unit 1200 having a first heating wire 1210 and a second heater electrode unit 2200 having a second heating wire 2210. The first sensor electrode unit 1300 is closer to the first heating wire 1210 than to the second heating wire 2210. The second sensor electrode unit 2300 is closer to the second heating wire 2210 than to the first heating wire 1210. At least one of the first sensor electrode unit 1300 and the second sensor electrode unit 2300 includes a first sensor electrode 1301, 2301 and a second sensor electrode 1302, 2302 spaced apart from the first sensor electrode 1301, 2301. The first sensor electrode 1301, 2301 is formed on a first side of the substrate 100, and the second sensor electrode 1302, 2302 is formed on a second side of the substrate 100. The substrate 100 is provided with an etching hole 103 penetrating from the first side of the substrate 100 to the second side of the substrate 100.

When anodizing a metallic base material, an anodized film is formed. The anodized film is composed of a porous layer having several pores on the surface (upper surface) and a barrier layer existing at the lower portion of the porous layer. Here, the metallic base material may be aluminum (Al), titanium (Ti), tungsten (W), zinc (Zn), etc. It is desired that the metallic base material is made of aluminum or aluminum alloy material that is lightweight, is easy to process, is excellent in thermal conductivity, and obviates concern about heavy metal contamination.

For example, when anodizing the surface of aluminum, an alumina film is formed. The alumina film is composed of a porous alumina layer having several pores 102 penetrating the surface (upper surface) in a vertical direction and a barrier layer existing at the lower portion of the porous alumina layer. According to the embodiment of the present invention, the substrate 100 may be, for example, an anodized film obtained by removing aluminum. Accordingly, the porous alumina layer is formed at the upper portion of the substrate 100, and the barrier layer is formed at the lower portion of the substrate 100. Alternatively, the substrate 100 may be composed of only the porous alumina layer through which the pore 102 penetrates in a vertical direction by removing the barrier layer of the alumina film.

The diameter of the pore 102 is formed in nano-meters. Also, the diameter of the pore 102 is smaller than the minimum lateral width of the heater electrode unit or the sensor electrode unit formed on the substrate 100. A part or all of the pores 102 may be blocked by the heater electrode unit or the sensor electrode unit. Due to the pores 102 placed at the lower portion of the heater electrode unit, the heat of the heater electrode unit is prevented from being transferred to the lower portion.

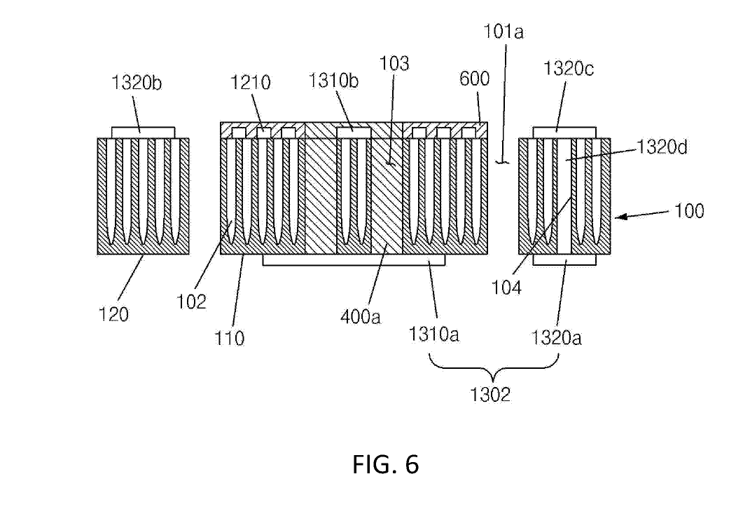

Hereinafter, as shown in FIG. 6, the description is based on a substrate 100 where only aluminum is removed.

By removing the aluminum from the anodized aluminum, the upper portion of the pore 102 of the substrate 100 is opened and the lower portion of the pore 102 is blocked by the barrier layer. As described above, the porous alumina layer is formed on the substrate 100, and thus heat capacity of the micro sensor is reduced.

The substrate 100 includes: two first supporting portion 110 formed in cylindrical shapes on both sides of the substrate 100; a second supporting unit 120 formed outside of the first supporting portion 110 being spaced apart from the first supporting portion 110; and several bridge portions 130 connecting the first supporting portions 110 and the second supporting unit 120. In the embodiment, two first supporting portions 110 are formed on one substrate 100, but one or three or more first supporting portions 110 may be formed. The first supporting portions 110 are spaced apart from each other.

Also, the substrate 100 is provided with the etching hole 103 penetrating from the first side of the substrate 100 to the second side of the substrate 100. In the embodiment, the etching hole 103 is formed by penetrating from the upper surface of the substrate 100 to the lower surface of the substrate 100. The etching hole 103 described below is formed by penetrating both the surface on which the first sensor wire 1310b, 2310a is formed and the surface on which the second sensor wire 1310a, 2310b is formed.

The etching hole 103 is formed on the central portion of the first supporting portion 110.

The maximum width of the etching hole 103 is wider than the maximum width of the pore 102. As described above, the width of the etching hole 103 is formed in micrometers, and thus the etching hole is easily inserted in the sensing material.

The etching hole 103 is formed at the circumference of the first sensor electrode 1301, 2301 described below. Specifically, the etching hole 103 is formed to surround the first sensor wire 1310b, 2310a of the first sensor electrode 1301, 2301.

Accordingly, the etching hole 103 is placed at the side of the first sensor wire 1310b, 2310a.

The etching hole 103 is formed in the arc shape at an area except for the portion supporting the first sensor wire 1310b, 2310a. Specifically, in the first supporting portion 110, the etching holes 103 are placed between the central portions supporting the first sensor wires 1310b and 2310a and portions supporting the first and second heating wires 1210 and 2210. The portions supporting the first sensor wires 1310b and 2310a and the portions supporting the first and second heating wires 1210 and 2210 are respectively connected to each other on the first side.

The etching hole 103 is formed between the first sensor wire 1310b, 2310a and the second sensor wire 1310a, 2310b. The etching hole 103 is formed to connect the first sensor wire 1310b, 2310a and the second sensor wire 1310a, 2310b via a sensing material described below.

A manufacturing method of the micro sensor where the etching hole 103 is formed will be described as follows.

A porous alumina layer (Anodic Aluminum Oxide, AAO) is formed on the substrate 100. Next, the sensor electrode unit and the heater electrode unit are formed on the substrate 100. Next, etching is performed on the substrate 100 by using etching liquid etching the porous alumina layer (Anodic Aluminum Oxide, AAO) of the substrate 100, and the etching hole 103 is formed as penetrating in a vertical direction between the first sensor wires 1310b and 2310a and the first and second heating wires 1210 and 2210. In this etching step, the sensor electrode unit and the heater electrode unit function as masks, whereby a manufacturing process is simple.

Several air gaps are formed near the first supporting portion 110, namely, between the first supporting portion 110 and the second supporting unit 120.

The air gap is placed at a side of the first supporting portion 110.

The air gap includes a first air gap 101a surrounding the first supporting portion 110 placed at the left and a second air gap 101b surrounding the first supporting portion 110 placed at the right.

Also, several air gaps are formed at the outer circumference of each first supporting portion 110. Several air gaps are discontinuously formed. Air gaps and bridge portions 130 are alternately placed around the first supporting portion 110. The bridge portions 130 are formed by forming air gaps through etching the periphery of the first supporting portion 110. Thus, a first end of each bridge portion 130 is connected to the first supporting portion 110 and a second end thereof is connected to the second supporting unit 120.

Hereinafter, the sensor electrode unit, the heater electrode unit, and an anti-etching dam 500 that are formed on the substrate will be disclosed.

The sensor electrode unit is formed on the substrate 100.

The sensor electrode unit detects gas by measuring the changes in the electrical characteristics when the gas is adsorbed onto the sensing material.

The sensor electrode unit includes the first sensor electrode unit 1300 and the second sensor electrode unit 2300.

At least one of the first sensor electrode unit 1300 and the second sensor electrode unit 2300 includes a first sensor electrode 1301, 2301 and a second sensor electrode 1302, 2302 spaced apart from the first sensor electrode 1301, 2301. In the embodiment, the first sensor electrode unit 1300 and the second sensor electrode unit 2300 respectively includes the first sensor electrodes 1301 and 2301 and the second sensor electrodes 1302 and 2302 spaced apart from the first sensor electrodes 1301 and 2301.

The first sensor electrodes 1301 and 2301 are formed on a first side of the substrate 100, and the second sensor electrodes 1302 and 2302 are formed on a second side of the substrate 100. In the embodiment, the first sensor electrodes 1301 and 2301 are formed on the upper surface of the substrate 100, and the second sensor electrodes 1302 and 2302 are formed on the lower surface of the substrate 100. That is, at the substrate 100, the second sensor electrode 1302 and 2302 are formed on a surface opposite to a surface on which the first sensor electrodes 1301 and 2301 are formed.

The first sensor electrode 1301, 2301 includes the first sensor wire 1310b, 2310a and the first sensor electrode pad 1320b, 2320a connected to the first sensor wire 1310b, 2310a.

The first sensor wires 1310b and 2310a are respectively formed on the upper surfaces of the first supporting portions 110 at the left and the right.

The first sensor wire 1310b, 2310a is formed in a straight line shape.

The first sensor electrode pad 1320b, 2320a is formed on the upper surface of the bridge portion 130 and the second supporting unit 120.

The second sensor electrode 1302, 2302 includes the second sensor wire 1310a, 2310b and the second sensor electrode pad 1320a, 2320b connected to the second sensor wire 1310a, 2310b.

The second sensor wires 1310a and 2310b are respectively formed on the lower surfaces of the first supporting portions 110 at the left and the right. The second sensor wires 1310a and 2310b are spaced apart from the first sensor wires 1310b and 2310a in a vertical direction.

When viewed in a plan view, an area of the second sensor wire 1310a, 2310b is larger than an area of the first sensor wire 1310b, 2310a.

The second sensor wire 1310a, 2310b of the second sensor electrode 1302, 2302 may block at least a part of the second side (lower portion) of the etching hole 103. In the embodiment, the second sensor wire 1310a, 2310b is formed in a circular plate shape and blocks the entire lower portion of the etching hole 103. The diameter of the second sensor wire 1310a, 2310b is larger than the external diameter of the etching hole 103.

The second sensor electrode pad 1320a, 2320b is formed on the lower surface of the bridge portion 130 and the second supporting unit 120.

An assistant pad 1320c, 2320c is formed on the first side, namely, the upper surface of the substrate 100. The assistant pad 1320c, 2320c is spaced from the first sensor electrode pad 1320b, 2320a.

The assistant pad 1320c, 2320c is placed above the second sensor electrode pad 1320a, 2320b.

The assistant pad 1320c, 2320c is connected to the second sensor electrode pad 1320a, 2320b.

Also, the second supporting unit 120 of the substrate 100 is provided with a pad hole 104 penetrating from the first side of the substrate 100 to the second side of the substrate 100 so as to connect the assistant pad 1320c, 2320c and the second sensor electrode pad 1320a, 2320b. The pad hole 104 is formed as penetrating in a vertical direction. The pad hole 104 is placed between the assistant pad 1320c, 2320c and the second sensor electrode pad 1320a, 2320b.

The horizontal cross-sectional area of the pad hole 104 is smaller than the horizontal cross-sectional area of the assistant pad 1320c, 2320c and of the second sensor electrode pad 1320a, 2320b. Therefore, the upper and the lower portions of the pad hole 104 are blocked by the assistant pad 1320c, 2320c and the second sensor electrode pad 1320a, 2320b.

A connecting bar 1320d is placed in the pad hole 104, and thus the assistant pad 1320c, 2320c and the second sensor electrode pad 1320a, 2320b are electrically connected to each other.

The widths of the first and second sensor electrode pads 1320b, 2320a and 1320a, 2320b are larger than the widths of the first and second sensor wires 1310b, 2310a and 1310a, 2310b.

The widths of the first and second sensor electrode pads 1320b, 2320a and 1320a, 2320b are wider towards the end portions.

The first sensor electrode unit 1300 and the second sensor electrode unit 2300 are formed of a mixture including one of or at least one of Pt, W, Co, Ni, Au, and Cu.

The assistant pad 1320c of the first sensor electrode unit 1300 is close to the end of the first sensor electrode pad 2320a of the second sensor electrode unit 2300. In the embodiment, the assistant pad 1320c of the first sensor electrode unit 1300 is spaced apart from the first sensor electrode pad 2320a of the second sensor electrode unit 2300.

Alternatively, the assistant pad 1320c of the first sensor electrode unit 1300 may be connected to the first sensor electrode pad 2320a of the second sensor electrode unit 2300. In this case, when a middle portion between the assistant pad 1320c of the first sensor electrode unit 1300 and the first sensor electrode pad 2320a of the second sensor electrode unit 2300 is used as a common electrode, the first sensor electrode unit 1300 and the second sensor electrode unit 2300 are connected to each other in parallel.

The heater electrode unit is formed on the upper surface of the substrate 100.

When the electrode is formed on the porous alumina layer of the alumina film, the pore 102 placed at the lower portion of the heater electrode unit and the sensor electrode unit has the upper portion and the lower portion thereof blocked by the heater electrode unit and the sensor electrode unit. As described above, the heater electrode unit is formed on the porous alumina layer, and thus the heat capacity of the micro sensor is small.

The heater electrode unit includes the first heater electrode unit 1200 and the second heater electrode unit 2200 spaced apart from the first heater electrode unit 1200.

The first heater electrode unit 1200 includes: the first heating wire 1210 closer to the first sensor wire 1310b than to the first sensor electrode pad 1320b; the first heater electrode unit pad 1220 formed on the second supporting unit 120 and the bridge portion 130 by being connected to the first heating wire 1210.

The first heating wire 1210 is formed on the first supporting portion 110 at the left, and surrounds at least a part of the first sensor wire 1310b. The first heater electrode unit pad 1220 includes a first heater electrode unit first pad 1220a and a first heater electrode unit second pad 1220b that are respectively connected to both ends of the first heating wire 1210. The first heater electrode unit first pad 1220a and the first heater electrode unit second pad 1220b are spaced apart from each other.

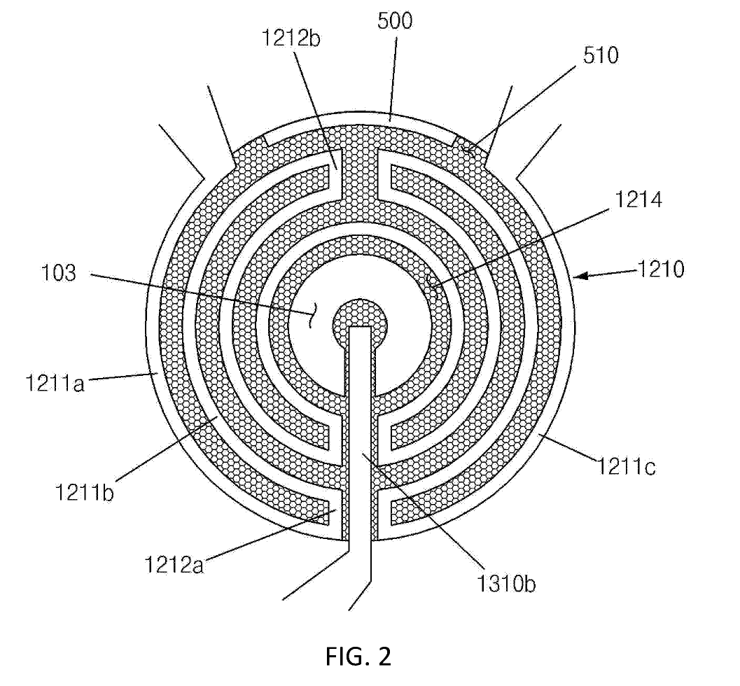

When viewed in a plan view like FIG. 2, the first heating wire 1210 is formed to be symmetrical about the vertical center line of the first supporting portion 110, and includes several arc portions formed in the arc shapes and several connecting portion connecting the arc portions.

The outermost side of the first heating wire 1210 is formed close to the edge of the first supporting portion 110.

The first heating wire 1210 includes: a first arc portion 1211a formed in an arc shape close to the first air gap 101a; a first connecting portion 1212a extending from an end of the first arc portion 1211a and bent toward the inside of the first supporting portion 110; a second arc portion 1211b in an arc shape extending from an end of the first connecting portion 1212a and spaced apart from the first arc portion 1211a inwards; a second connecting portion 1212b extending from an end of the second arc portion 1211b toward the inside of the first supporting portion 110; and a third arc portion 1211c. In this manner, several arc portions and connecting portions are formed by being repeatedly connected to each other.

The first heating wire 1210 is connected from the first arc portion 1211a to the third arc portion 1211c to have an integral body, and is symmetrical about the vertical center line of the first supporting portion 110 at the left.

As shown in FIG. 2, several arc portions of the first heating wire 1210 are formed in about the half-arc shape, and are symmetrically formed. Thus, the first heating wire 1210 is overall in a circular shape. Accordingly, temperature uniformity of the first supporting portion 110 may be enhanced.

The central portion of the first heating wire 1210 is a point where the left and right arc portions meet, and the central portion is in a lower side opened circular shape by joining two arc portions in arc shapes together. An isolated space portion 1214 is formed inside of the central portion. The isolated space portion 1214 is formed by extending from the central portion of the first heating wire 1210 to the front of the first heating wire 1210. That is, to form the isolated space portion 1214 from the central portion to the front of the first heating wire 1210, the left and right arc portions are spaced apart from each other. The first sensor wire 1310b is placed at the isolated space portion 1214. Accordingly, the first heating wire 1210 surrounds the rear and both sides of the first sensor wire 1310b.

Also, a second end portion of the first arc portion 1211a is connected to the first heater electrode unit second pad 1220b, and a first end portion of the third arc portion 1211c is connected to the first heater electrode unit first pad 1220a.

The first heater electrode unit 1200 is formed of a mixture including one or at least one of Pt, W, Co, Ni, Au, and Cu.

In the meantime, an anti-etching dam 500 is formed between both ends of the first heating wire 1210, namely, the ends of the first arc portion 1211a and the third arc portion 1211c respectively connected to the first heater electrode unit first pad 1220a and the first heater electrode unit second pad 1220b.

The anti-etching dam 500 at the left is formed in the arc shape between the first heating wire 1210 and the first air gap 101a. The anti-etching dam 500 is spaced apart from the first heating wire 1210 adjacent thereto.

The anti-etching dam 500 is close to the edge of the first supporting portion 110.

It is desired that the anti-etching dam 500 is formed outside of the first heating wire 1210 and is a metal. The material of the anti-etching dam 500 may be the same as the electrode material, and the electrode material may be a metal such as platinum, aluminum, copper, etc.

As shown in FIG. 2, the lengths of the first arc portion 1211a and the third arc portion 1211c are short, compared to the remaining arc portions located inside thereof. At the outer circumference of the first heating wire 1210, a space 510 is defined between the ends of the first arc portion 1211a and the third arc portion 1211c. The anti-etching dam 500 is placed at the space 510. The width of the anti-etching dam 500 is formed the same as or similar to the width of the first heating wire 1210.

The space 510 at the outer circumference of the first heating wire 1210 is partially filled by the area of the anti-etching dam 500. Thus, when viewed in a plan view, the outer circumference of the first heating wire 1210 and the anti-etching dam 500 is in a circular shape, whereby temperature uniformity of the first supporting portion 110 may enhance.

Also, by forming the anti-etching dam 500 at the space 510 between the ends of the first arc portion 1211a and the third arc portion 1211c, the bridge portion 130 may be designed to make the structure of the substrate 100 more stable. Also, it is easy to form the overall shape of the first supporting portion 110 in a circular shape. Accordingly, the position of the bridge portion 130 connecting the first supporting portion 110 and the second supporting unit 120 may be designed considering the stability of the entire structure of the micro sensor.

The anti-etching dam 500 prevents a space 510 of the first supporting portion 110 from being damaged by the etching liquid when forming the air gap 101 through etching. That is, the anti-etching dam 500 is formed close to the first heating wire 1210 formed on the first supporting portion 110, and prevents a particular shape (for example, the circular shape) of the first supporting portion 110 supporting the first heating wire 1210 from being damaged.

The widths of the first heater electrode unit first pad 1220a and the first heater electrode unit second pad 1220b are wider towards the outside. That is, the width of the first heater electrode unit pad 1220 is narrower towards the first heating wire 1210. The first heater electrode unit pad 1220 has a width wider than that of the first heating wire 1210.

The distance between the first heater electrode unit first pad 1220a and the first supporting portion 110 at the right is shorter than the distance between the first heater electrode unit second pad 1220b and the first supporting portion 110 at the right.

The second heater electrode unit 2200 is similar to the first heater electrode unit 1200.

The second heater electrode unit 2200 includes: the second heating wire 2210 closer to the first sensor wire 2310a than to the first sensor electrode pad 2320a; and the second heater electrode unit pad 2220 formed on the second supporting unit 120 and the bridge portion 130 be being connected to the second heating wire 2210.

The second heating wire 2210 is formed on the upper surface of the first supporting portion 110 at the right.

Thus, the first sensor wire 1310b and the first heating wire 1210 are formed on the upper surface of the first supporting portion 110 at the left, the first sensor wire 2310a and the second heating wire 2210 are formed on the upper surface of the first supporting portion 110 at the right.

Accordingly, the first sensor wire 1310b of the first sensor electrode unit 1300 is closer to the first heating wire 1210 than to the second heating wire 2210. The first sensor wire 2310a of the second sensor electrode unit 2300 is closer to the second heating wire 2210 than to the first heating wire 1210.

Also, the first heating wire 1210 and the second heating wire 2210 have different heating values.

For different heating values of the first heating wire 1210 and the second heating wire 2210, as shown in FIG. 1, the first heating wire 1210 and the second heating wire 2210 may have different lengths, or the first heating wire 1210 and the second heating wire 2210 may have different thicknesses.

In the embodiment, the length of the first heating wire 1210 is longer than the length of the second heating wire 2210, and thus the first sensing material 400a formed on the upper portion of the first supporting portion 110 at the left may be heated is to a higher temperature than that of the second sensing material 400b formed on the upper portion of the first supporting portion 110 at the right. Accordingly, the first sensor electrode unit 1300 and the second sensor electrode unit 2300 may detect different kinds of gas.

The gaps of the first heating wire 1210 are narrower than the gaps of the second heating wire 2210 such that the first heating wire 1219 is more bent, whereby the first heating wire 1210 and the second heating wire 2210 may have different lengths in a limited space (first supporting portion).

Also, different from the embodiment, the both sides of the first heating wire and the second heating wire may be not symmetrical about the vertical center line or the horizontal center line of the first supporting portion 110. That is, the first heating wire and/or the second heating wire may be formed by connecting two heating wires bent in different shapes in series.

The second heating wire 2210 is formed on the first supporting portion 110 at the right, and surrounds at least a part of the first sensor wire 2310a of the second sensor electrode unit 2300. The second heater electrode unit pad 2220 includes a second heater electrode unit first pad 2220a and a second heater electrode unit second pad 2220b that are respectively connected to both ends of the second heating wire 2210. The second heater electrode unit first pad 2220a and the second heater electrode unit second pad 2220b are spaced apart from each other.

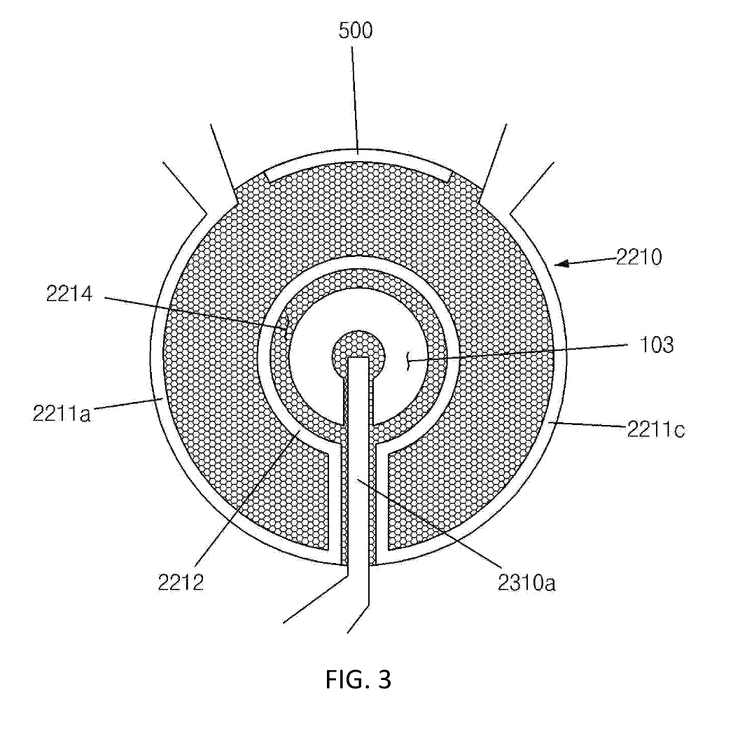

When viewed in a plan view like FIG. 3, the second heating wire 2210 is formed to be symmetrical about the vertical center line of the first supporting portion 110 at the right.

The outer most side of the second heating wire 2210 is formed close to the edge of the first supporting portion 110.

The second heating wire 2210 includes a first arc portion 2211a and a third arc portion 2211c formed in arc shapes and closed to the second air gap 101b, and a sensor wire surrounding portion 2212 formed between the first arc portion 2211a and the third arc portion 2211c.

The first arc portion 2211a is connected to the second heater electrode unit first pad 2220a, and the third arc portion 2211c is connected to the second heater electrode unit second pad 2220b.

The sensor wire surrounding portion 2212 is connected to the front ends of the first arc portion 2211a and of the third arc portion 2211c, and is bent to surround the first sensor wire 2310a. Accordingly, the sensor wire surrounding portion 2212 is provided with the isolated space portion 2214 having the opened front.

An anti-etching dam 500 is formed in an arc shape between both ends of the second heating wire 2210, namely, the ends of the first arc portion 2211a and the third arc portion 2211c respectively connected to the second heater electrode unit first pad 2220a and the second heater electrode unit second pad 2220b.

The anti-etching dam 500 is placed between the second heater electrode unit first pad 2220a and the second heater electrode unit second pad 2220b.

The anti-etching dam 500 formed on the upper surface of the first supporting portion 110 at the right and the anti-etching dam 500 formed on the upper surface of the first supporting portion 110 at the left have the same or similar shapes and effects, and thus the detailed description thereof will be omitted.

As described above, the anti-etching dam 500 is formed on the first supporting portion 110 of the substrate 100 between the first air gap 101a and the first heating wire 1210. The anti-etching dam 500 is formed on the first supporting portion 110 of the substrate 100 between the second air gap 101b and the second heating wire 2210.

The widths of the second heater electrode unit first pad 2220a and the second heater electrode unit second pad 2220b are wider towards the outside. That is, the width of the second heater electrode unit pad 2220 is narrower towards the second heating wire 2210, and the width of the second heater electrode unit pad 2220 is wider than the width of the second heating wire 2210.

The distance between the second heater electrode unit first pad 2220a and the is shorter than the distance between the second heater electrode unit second pad 2220b and the first supporting portion 110 at the left.

The second heater electrode unit first pad 2220a is close to the first heater electrode unit first pad 1220a. In the embodiment, the second heater electrode unit first pad 2220a is spaced apart from the first heater electrode unit first pad 1220a.

As described above, the second heater electrode unit first pad 2220a is separated from the first heater electrode unit first pad 1220a. The assistant pad 1320c of the first sensor electrode unit 1300 is separated from the first sensor electrode pad 2320a of the second sensor electrode unit 2300. Accordingly, the left and right sensors can be individually controlled. Therefore, depending on the situation, only the left sensor is turned on to detect gas, or only the right sensor is turned on to detect gas.

Different from the above description, the second heater electrode unit first pad 2220a may be connected to the first heater electrode unit first pad 1220a. In this case, when a middle portion between the second heater electrode unit first pad 2220a and the first heater electrode unit first pad 1220a is used as a common electrode, the first heating wire 1210 and the second heating wire 2210 are connected to each other in parallel. Alternatively, when simply connecting the second heater electrode unit first pad 2220a and the first heater electrode unit first pad 1220a without using the middle portion therebetween as a common electrode and applying electricity to the first heater electrode unit 1200 or to the second heater electrode unit 2200, the first heating wire 1210 and the second heating wire 2210 are connected to each other in series. As described above, depending on position in the heater electrode unit to which electricity is applied, the first heating wire 1210 and the second heating wire 2210 are connected to each other in parallel or in series.

A passivation layer 600 is formed on at least some surfaces (the upper surface and the side surface) of the first heating wire 1210 and the second heating wire 2210 of the heater electrode unit. The passivation layer 600 may be formed of oxide type material. Moreover, the passivation layer 600 may be formed of at least one of tantalum oxide (TaOx), titanium oxide (TiO₂), silicon oxide (SiO₂), and aluminum oxide (Al₂O₃).

The passivation layer 600 is formed on the upper surface of the first supporting portion 110. The passivation layer 600 is formed to cover the upper portions and the side portions of the first heating wire 1210 and the second heating wire 2210. The passivation layer 600 surrounds the perimeter of the first and second sensing materials 400a and 400b. Accordingly, the passivation layer 600 is formed in a ring shape. The passivation layer 600 covers a part of the first sensor wire 1310b, 2310a.

Also, the first air gap 101a surrounds the first heating wire 1210, and the second air gap 101b surrounds the second heating wire 2210. As described above, at least one air gap is formed between the first heating wire 1210 and the second heating wire 2210.

The first air gap 101a and the second air gap 101b are formed in the same shape.

The widths of the first and second air gaps 101a and 101b are wider than the maximum width of the pore 102, and wider than the maximum widths of the first and second heating wires 1210 and 2210. The first and second air gaps 101a and 101b is formed corresponding to the shape of the first and second heating wires 1210 and 2210 and the shape of the first supporting portion 110. The first and second air gaps 101a and 101b are formed in arc shapes, and are formed in threes. The several first and second air gaps 101a and 101b are spaced apart from each other in a circumferential direction. That is, several first and second air gaps 101a and 101b are discontinuously formed.

Specifically, the first air gaps 101a are placed between the first sensor electrode pad 1320b and the first heater electrode unit second pad 1220b, between the first heater electrode unit second pad 1220b and the first heater electrode unit first pad 1220a, and between the first heater electrode unit first pad 1220a and the first sensor electrode pad 1320b.

The second air gaps 101b are placed between the second heater electrode unit second pad 2220b and the second heater electrode unit first pad 2220a, between the second heater electrode unit first pad 2220a and the first sensor electrode pad 2320a, and between the first sensor electrode pad 2320a and the second heater electrode unit second pad 2220b.

That is, the first and second air gaps 101a and 101b are formed at an area except for the portion supporting the first and second heater electrode units 1200 and 2200 and the first and second sensor electrode units 1300 and 2300.

The first and second air gaps 101a and 101b are formed penetrating in vertical directions. That is, the first and second air gaps 101a and 101b are spaces formed by penetrating from the upper surface to the lower surface of the substrate 100.

Due to the first and second air gaps 101a and 101b, on the substrate 100, formed are the first supporting portion 110 at the left supporting the first heating wire 1210 and the first and second sensor wires 1310b and 1310a in common; the first supporting portion 110 at the right supporting the second heating wire 2210 and the first and second sensor wires 2310a and 2310b in common; and the second supporting unit 120 and the bridge portion 130 that support the first and second heater electrode unit pads 1220 and 2220, the first and second sensor electrode pads 1320b, 2320a and 1320a, 2320b, and the assistant pads 1320c and 2320c.

Each first supporting portion 110 is wider than the total area of the heating wire and the sensor wire formed on the first supporting portion 110.

The first supporting portion 110 and the second supporting unit 120 are spaced apart from each other due to the air gaps at the portions other than the bridge portions 130. Accordingly, the first supporting portion 110 and the second supporting unit 120 are connected to each other at three portions due to three bridge portions 130 as shown in FIG. 1.

The second supporting unit 120 is placed between the first air gap 101a and the second air gap 101b that are placed between the first supporting portions 110 at the left and the right. Accordingly, the first supporting portion 110 at the left, the first air gap 101a, the second supporting unit 120, the second air gap 101b, and the first supporting portion 110 at the right are arranged from the left to the right direction in order.

Alternatively, the air gap may include the first air gap 101a and the third air gap coupled to the second air gap 101b. The third air gap may be placed between the first heating wire 1210 and the second heating wire 2210.

At the central portions of the first supporting portions 110, the first and second sensing materials 400a and 400b are formed. The first and second sensing materials 400a and 400b are formed at the upper portion and the side portion of the first sensor wire 1310b, 2310a, at the inside of the etching hole 103, and the upper portion of the second sensor wire 1310a, 2310b. That is, the first and second sensing materials 400a and 400b are placed between the first sensor wires 1310b and 2310a and the second sensor wires 1310a and 2310b.

As described above, the first and second sensing materials 400a and 400b are in contact with the surfaces of the first sensor wire 1310b, 2310a and of the second sensor wire 1310a, 2310b, and are placed at a space (gap) between the first sensor wire 1310b, 2310a and the second sensor wire 1310a, 2310b.

The first and second sensing materials 400a and 400b are formed to cover the first sensor wire 1310b, 2310a and the second sensor wire 1310a, 2310b.

The upper surfaces of the first and second sensing materials 400a and 400b are exposed to the outside.

The first sensing material 400a and the second sensing material 400b may be formed of the same material or different materials. Even with the same sensing material, adsorbed gas may differ depending on the heated temperature.

Hereinafter, the operation of the embodiment having the above-described configuration will be disclosed.

In order to measure the gas concentration, first, the same electric power is simultaneously applied to the first heater electrode unit pad 1220 and to the second heater electrode unit pad 2220 so as to heat the first heating wire 1210 and the second heating wire 2210. The first heating wire 1210 is longer than the second heating wire 2210, and thus the first sensing material 400a is heated to a higher temperature than that of the second sensing material 400b.

Different kinds of gas are adsorbed onto or desorbed from the first and second sensing materials 400a and 400b heated to different temperatures.

Accordingly, electrical conductivity between the first sensor wire 1310b, 2310a and the second sensor wire 1310a, 2310b changes, and the changes in the electrical conductivity is measured to detect gas.

Through these processes, the micro sensor of the embodiment can simultaneously detect several kinds of gas.

As described above, although the exemplary embodiments of the present invention have been disclosed, those skilled in the art will appreciate that various modifications or changes are possible, without departing from the scope and spirit of the invention as disclosed in the accompanying claims.

What is claimed is:

1. A micro sensor comprising:
a substrate; and
a sensor electrode unit provided on the substrate,
wherein the sensor electrode unit includes a first sensor electrode and a second sensor electrode spaced apart from the first sensor electrode,
the first sensor electrode is provided on a first side of the substrate,
the second sensor electrode is provided on a second side of the substrate, and
the substrate is provided with an etching hole penetrating from the first side to the second side of the substrate.

2. The micro sensor of claim 1, wherein the etching hole is provided at a circumference of the first sensor electrode.

3. The micro sensor of claim 1, wherein the second sensor electrode blocks at least a part of the etching hole on the second side.

4. The micro sensor of claim 1, wherein the first sensor electrode includes a first sensor wire,
the second sensor electrode includes a second sensor wire, and
an area of the second sensor wire is larger than an area of the first sensor wire.

5. The micro sensor of claim 1, wherein the first sensor electrode includes a first sensor wire and a first sensor electrode pad connected to the first sensor wire,
the second sensor electrode includes a second sensor wire and a second sensor electrode pad connected to the second sensor wire,
the first side of the substrate is provided with an assistant pad, and
the assistant pad is connected to the second sensor electrode pad.

6. The micro sensor of claim 5, wherein the substrate is provided with a pad hole penetrating from the first side to the second side of the substrate so as to connect the assistant pad and the second sensor electrode pad to each other.

7. The micro sensor of claim 1, further comprising:
a heater electrode unit provided on the first side of the substrate.

8. The micro sensor of claim 7, wherein the substrate is an anodized film obtained by anodizing a metallic base material and removing the base material.

9. The micro sensor of claim 7, wherein the substrate is provided with an air gap surrounding the heater electrode unit.

10. The micro sensor of claim 7, wherein at least a part of a surface of the heater electrode unit is provided with a passivation layer.

11. A micro sensor comprising:
a substrate;
a sensor electrode unit provided on the substrate; and
a heater electrode unit provided on the substrate,
wherein the sensor electrode unit includes a first sensor electrode unit and a second sensor electrode unit,
the heater electrode unit includes a first heater electrode unit having a first heating wire and a second heater electrode unit having a second heating wire,
the first sensor electrode unit is closer to the first heating wire than to the second heating wire,
the second sensor electrode unit is closer to the second heating wire than to the first heating wire,
at least one of the first sensor electrode unit and the second sensor electrode unit includes a first sensor electrode and a second sensor electrode spaced apart from the first sensor electrode,
the first sensor electrode is provided on a first side of the substrate,
the second sensor electrode is provided on a second side of the substrate, and
the substrate is provided with an etching hole penetrating from the first side to the second side of the substrate.

12. The micro sensor of claim 11, wherein the first heating wire and the second heating wire have different heating values.
